(19) United States
(12) Patent Application Publication
(10) Pub. No.: US 2014/0256995 A1
(43) Pub. Date: Sep. 11, 2014

(54) PROCESS FOR PRODUCING 2,3,3,3-TETRAFLUOROPROPENE

(71) Applicant: HONEYWELL INTERNATIONAL INC., Morristown, NJ (US)

(72) Inventors: Haiyou Wang, Amherst, NY (US); Selma Bektesevic, Williamsville, NY (US); Hsueh Tung, Getzville, NY (US)

(73) Assignee: HONEYWELL INTERNATIONAL INC., Morristown (US)

(21) Appl. No.: 14/348,754

(22) PCT Filed: Sep. 29, 2012
(86) PCT No.: PCT/US2012/058152

§ 371 Date: Mar. 31, 2014

Related U.S. Application Data
(60) Provisional application No. 61/541,656, filed on Sep. 30, 2011.

(51) Int. Cl. C07C 17/20; C07C 21/04

(52) U.S. Cl. CPC C07C 17/20; C07C 21/04
USPC 570/160; 570/189

(57) ABSTRACT

The present invention relates, in part, to the discovery that the presence of moisture in 1,1,2,3-tetrachloropropene (HCO-1230xa) results in catalyst deactivation and accelerated corrosion in the reactor during the fluorination of HCO-1230xa to 2-chloro-3,3,3-trifluoropropene. By substantially removing the moisture, it is shown that the catalyst life is extended and results in improved operation efficiency of the fluorination reaction. Such steps similarly result in an overall improvement in the production of certain hydrofluoroolefins, particularly 2,3,3,3-tetrafluoropropene (HFO-1234yf).

### CROSS REFERENCE TO RELATED APPLICATIONS

This application claims priority of U.S. provisional application U.S. Ser. No. 61/541,656, which was filed on Sep. 30, 2011.

### FIELD OF THE INVENTION

The present invention relates to a process for preparing fluorinated organic compounds, more particularly to a process for preparing fluorinated olefins, and even more particularly to a process for producing 2,3,3,3-tetrafluoropropene (HFO-1234yf).

### BACKGROUND OF THE INVENTION

Hydrofluoroolefins (HFOs), such as tetrafluoropropenes (including 2,3,3,3-tetrafluoropropene (HFO-1234yf)), are now known to be effective refrigerants, fire extinguishants, heat transfer media, propellants, foaming agents, blowing agents, gaseous dielectrics, sterilant carriers, polymerization media, particulate removal fluids, carrier fluids, buffing abrasive agents, displacement drying agents and power cycle working fluids. Unlike chlorofluorocarbons (CFCs) and hydrochlorofluorocarbons (HCFCs), both of which potentially damage the Earth's ozone layer, HFOs do not contain chlorine and, thus, pose no threat to the ozone layer. HFO-1234yf has also been shown to be a low global warming compound with low toxicity and, hence, can meet increasingly stringent requirements for refrigerants in mobile air conditioning. Accordingly, compositions containing HFO-1234yf are among the materials being developed for use in many of the aforementioned applications.

Several methods of preparing HFOs are known. For example, U.S. Pat. No. 4,900,874 (Ihara et al) describes a method of making fluorine containing olefins by contacting hydrogen gas with fluorinated alcohols. Although this appears to be a relatively high-yield process, commercial scale handling of hydrogen gas at high temperature is hazardous. Also, the cost of commercially producing hydrogen gas, such as building an on-site hydrogen plant, is economically costly.

U.S. Pat. No. 2,931,840 (Marquis) describes a method of making fluorine containing olefins by pyrolysis of methyl chloride and tetrafluoroethylene or chlorodifluoromethane. This process is a relatively low yield process and a very large percentage of the organic starting material is converted to unwanted and/or unimportant byproducts, including a sizeable amount of carbon black which tends to deactivate the catalyst used in the process.

The preparation of HFO-1234yf from trifluoroacetylacetone and sulfur tetrafluoride has been described (See Banks, et al., Journal of Fluorine Chemistry, Vol. 82, Iss. 2, p. 171-174 (1997)). Also, U.S. Pat. No. 5,162,594 (Krespan) discloses a process wherein tetrafluoroethylene is reacted with another fluorinated ethylene in the liquid phase to produce a polyfluoroolefin product.

The preparation of HFO-1234yf is also described in U.S. Pat. Nos. 8,084,653, 8,071,825 and 8,058,486, the contents of which are incorporated by reference.

However, there remains a need for an economic means of producing hydrofluoroolefins, such as HFO-1234yf. The present invention satisfies this need among others.

### SUMMARY OF INVENTION

The present invention relates, in part, to the surprising discovery that the presence of moisture in certain vaporized starting or intermediate feed streams used for the production of certain HFOs, such as 2,3,3,3-tetrafluororpropene (HFO-1234yf), can promote the formation of both oxidized oligomers and solid inorganic salts. This, in turn, results in the deactivation of catalysts used in the initial fluorination step for HFO production. Accordingly, in one aspect, the present invention provides one or more process steps for removing moisture from the feed streams so as to prolong the catalyst life and improve the reaction efficiency.

In one aspect, the present invention relates to a feed stock for use in preparing a fluororolefin, where the feed stock includes a composition of 1,1,2,3-tetrachloropropene that is substantially free of water. While the definition of “substantially free” may be any provided herein, in one aspect the water content is less than about 200 ppm of water; less than about 100 ppm of water; or less than about 50 ppm of water.

In another aspect, the present invention relates to a method for reducing the moisture content of a 1,1,2,3-tetrachloropropene feed stock by providing a composition comprising 1,1,2,3-tetrachloropropene; and reducing the moisture content of the composition such that it is substantially free of water. The moisture content may be reduced using distillation, and/or using one or more dessicants. Dessicants may include, but are not limited to, silica gel, activated charcoal, calcium sulfate, calcium chloride, montmorillonite clay, a molecular sieve, and combinations thereof.

In another aspect, the present invention relates to a process for preparing 2-chloro-3,3,3-trifluoropropene by providing a starting composition comprising at least one compound of formula I

CX₂═CCl—CH₂X (I)

wherein X is independently selected from F, Cl, Br, and I, provided that at least one X is not fluorine and wherein the starting composition is substantially free of water; and contacting said starting composition with a fluorinating agent to produce a final composition comprising 2-chloro-3,3,3trifluoropropene. In certain embodiments, at least one compound of formula I has at least one X is a chlorine. In further embodiments, at least one compound of formula I has a chlorine at each X position. In even further embodiments, at least one compound of formula I comprises 1,1,2,3-tetrachloropropene.

The step of contacting the starting composition with a fluorinating agent may occur in the presence of a catalyst. In one aspect, the contacting steps occur in a vapor phase with or without the presence of a vapor phase catalyst. Vapor phase catalysts used for such a reaction include, but are not limited to, a chromium oxide, a chromium hydroxide, a chromium halide, a chromium oxyhalide, an aluminum oxide, an aluminum hydroxide, an aluminum halide, an aluminum oxyhalide, a cobalt oxide, a cobalt hydroxide, a cobalt halide, a cobalt oxyhalide, a manganese oxide, a manganese hydroxide, a manganese halide, a manganese oxyhalide, a nickel oxide, a nickel hydroxide, a nickel halide, a nickel oxyhalide, an iron oxide, an iron hydroxide, an iron halide, an iron oxyhalide, inorganic salts thereof, fluorinated derivatives thereof and combinations thereof. In certain embodiments, the catalyst comprises a chromium oxide, such as, but not limited to, Cr₂O₃.

In even further aspects, the present invention relates to a process for preparing 2,3,3,3-tetrafluoroprop-1-ene by
a. providing a starting composition comprising a compound of formula I

CX₂═CCl—CH₂X (I)

wherein X is independently selected from F, Cl, Br, and I, provided that at least one X is not fluorine and the starting composition is substantially free of water;
b. contacting the starting composition with a first fluorinating agent to produce a first intermediate composition including 2-chloro-3,3,3-trifluoropropene and a first chlorine-containing byproduct;
c. contacting the first intermediate composition with a second fluorinating agent to produce a second intermediate composition including 2-chloro-1,1,1,2-tetrafluoropropane; and
d. dehydrochlorinating at least a portion of the 2-chloro-1,1,1,2-tetrafluoropropane to produce a reaction product including 2,3,3,3-tetrafluoroprop-1-ene.

Additional embodiments and advantages to the present invention will be readily apparent to one of skill in the art, based on the disclosure provided herein.

### BRIEF DESCRIPTION OF THE DRAWING

These and other features, aspects, and advantages of the present invention will become better understood with regard to the following description, appended claims, and accompanying drawings.

FIG. 1 depicts graphically the amount of product, HCFO-1233xf produced in accordance with the procedure in Example 4 as a function of time on stream during the reaction of HCO-1230xa to HCFO-1233xf.

### DETAILED DESCRIPTION OF THE INVENTION

According to one embodiment, the present invention comprises a manufacturing process for making 2,3,3,3-tetrafluoroprop-1-ene using a starting material according to formula I:

CX₂═CCl—CH₂X (Formula I)

wherein X is independently selected from F, Cl, Br, and I, provided that at least one X is not fluorine. In certain embodiments, the compound(s) of Formula I contain at least one chlorine, more preferably a majority of X is chlorine, and even more preferably all Xs are chlorine. In certain embodiments, the compound of formula I is 1,1,2,3-tetrachloropropene (HCO-1230xa).

The method generally comprises at least three reaction steps. In the first step, a starting composition of Formula I (such as 1,1,2,3-tetrachloropropene) is reacted with anhydrous HF in a first vapor phase reactor (fluorination reactor) to produce a mixture of 2-chloro-3,3,3-trifluoropropene (HCFO-1233xf) and HCl. In certain embodiments, the reaction occurs in the vapor phase in the presence of a vapor phase catalyst, such as, but not limited to, a fluorinated chromium oxide. The catalyst may (or may not) have to be activated with anhydrous hydrogen fluoride HF (hydrogen fluoride gas) before use depending on the state of the catalyst.

While fluorinated chromium oxides are disclosed as the vapor phase catalyst, the present invention is not limited to this embodiment. Any fluorination catalysts known in the art may be used in this process. Suitable catalysts include, but are not limited to chromium, aluminum, cobalt, manganese, nickel and iron oxides, hydroxides, halides, oxyhalides, inorganic salts thereof and their mixtures. Combinations of catalysts suitable for the present invention nonexclusively include Cr₂O₃, FeCl₃/C, Cr₂O₃/Al₂O₃, Cr₂O₃/AlF₃, Cr₂O₃/carbon, CoCl₂/Cr₂O₃/Al₂O₃, NiCl₂/Cr₂O₃/Al₂O₃, CoCl₂/AlF₃, NiCl₂/AlF₃ and mixtures thereof. Chromium oxide/aluminum oxide catalysts are described in U.S. Pat. No. 5,155,082 which is incorporated herein by reference. Chromium (III) oxides such as crystalline chromium oxide or amorphous chromium oxide are preferred with amorphous chromium oxide being most preferred. Chromium oxide (Cr₂O₃) is a commercially available material which may be purchased in a variety of particle sizes. Fluorination catalysts having a purity of at least 98% are preferred. The fluorination catalyst is present in an excess but in at least an amount sufficient to drive the reaction.

Prior to the reaction, the compound of formula I, particularly when it is HCO-1230xa, is first purified to form a starting feed stream that is substantially free of moisture or water. While commercially available anhydrous HF is normally substantially water free, high level of moisture can be found in HCO-1230xa. Typically, the compounds of Formula I, and HCO-1230xa are used without reducing the amount of water. As used herein, the term “substantially free” means the reduction of moisture or water content within the feed stock of a sufficient volume to improve the catalyst life and process efficiency, as compared to the catalyst life or process efficiency when the moisture or water is not removed. In certain embodiments, the term about refers to plus or minus 10% ppm. In certain embodiments, the moisture or water content is less than about 200 ppm, in further embodiments it is less than about 100 ppm, in even further embodiments it is less than about 50 ppm.

In an embodiment, the moisture content of the compound of Formula I, e.g., HCO-1230xa, and/or a composition containing same is less than about 190 ppm, while in another embodiment, it is less than about 180 ppm, and in another embodiment, it is less than about 170 ppm. In other embodiments of the present invention, the moisture content of the compound of Formula I, e.g., HCO-1230xa, and/or a composition containing same is less than about 160 ppm; is less than about 150 ppm; is less than about 140 ppm; is less than about 130 ppm; is less than about 120 ppm; is less than about 110 ppm; is less than about 100 ppm; is than about 90 ppm; is less than about 80 ppm; is less than about 70 ppm; is less than about 60 ppm; is less than about 50 ppm; is less than about 40 ppm; is less than about 30 ppm; is less than about 20 ppm. In other embodiments, the moisture content of the compound of Formula I, e.g., HCO-1230xa, and/or a composition containing same ranges from about 10 ppm to about 200 ppm, while in other embodiments, it ranges from about 10 ppm to about 150 ppm, while in still other embodiments, it ranges from about 11 ppm to about 100, while in another embodiment, it ranges from about 12 ppm to about 50 ppm. The present invention contemplates a moisture content of the compound of Formula I, e.g. HCO-1230xa and/or a composition containing same of 100 ppm, 99 ppm, 98 ppm, 97 ppm, 96 ppm, 95 ppm, 94 ppm, 93 ppm, 92 ppm, 91 ppm, 90 ppm, 89 ppm, 88 ppm, 87 ppm, 86 ppm, 85 ppm, 84 ppm, 83 ppm, 82 ppm, 81 ppm, 80 ppm, 79 ppm, 78 ppm, 77 ppm, 76 ppm, 75 ppm, 74 ppm, 73 ppm, 72 ppm, 71 ppm, 70 ppm, 69 ppm, 68 ppm, 67 ppm, 66 ppm, 65 ppm, 64 ppm, 63 ppm, 62 ppm, 61 ppm, 60 ppm, 59 ppm, 58 ppm, 57 ppm, 56 ppm, 55 ppm, 54 ppm, 53 ppm, 52 ppm, 51 ppm, 50 ppm, 49 ppm, 48 ppm, 47 ppm, 46 ppm, 45 ppm, 44 ppm, 43 ppm, 42 ppm, 41 ppm, 40 ppm, 39 ppm, 38 ppm, 37 ppm, 36 ppm, 35 ppm, 34 ppm, 33 ppm, 32 ppm, 31 ppm, 30 ppm, 29 ppm, 28 ppm, 27 ppm, 26 ppm, 25 pm, 24 ppm, 23 ppm, 22 ppm, 21 ppm, 20 ppm, 19 ppm, 18 ppm, 17 ppm, 16 ppm, 15 ppm, 14 ppm 13 ppm, 12 ppm, 11 ppm, 10 ppm, and even lower.

Any conventional technique can be used to remove moisture. Non-limiting techniques include distillation, and/or absorption using desiccants, and/or the like. Distillation can be operated at atmospheric pressure, super-atmospheric pressure or under vacuum and can be performed using standard distillation methods for separating two compounds. In addition, the water may be separated out by distillation. Another method of removing the moisture from the compound of Formula I, e.g., HCO-1230xa, and/or composition containing same is by the use of dessicants, whereby the dessicant is in contact with the compound of Formula I, e.g., HCO-1230xa, and/or composition containing same for sufficient amount of time to reduce the moisture content thereof so that it is substantially free of water. While various desiccants can be used in a variety of ways, in certain embodiments the compound of Formula I, e.g., HCO-1230xa or composition containing same, is dried in pre-packaged desiccant in continuous mode. Non-limiting desiccants include silica gel, activated charcoal, calcium sulfate, calcium chloride, montmorillonite clay, and various molecular sieves. Once the feed is substantially free from moisture, HF (hydrogen fluoride) and the moisture free starting feed are then fed continuously to a vaporizer and the vaporized reactants to the catalyst bed.

The moisture content of the compound of Formula. I, e.g., HCO-1230xa, and/or composition containing same is measured by conventional means, such as Karl Fischer titration and the like.

When the compound of formula I is HCO-1230xa, the molar ratio of HF to HCO-1230xa in step 1 of the reaction is 1:1 to 1:50 and, in certain embodiments, from about 1:10 to about 1:20. The reaction between HF and HCO-1230xa is carried out at a temperature from about 150° C. to about 400° C. (in certain embodiments, about 180° C. to about 300° C.) and at a pressure of about 0 psig to about 200 psig (in certain embodiments from about 0 psig to about 100 psig). Contact time of the HCO-1230xa with the catalyst may range from about 1 second to about 60 seconds, however, longer or shorter times can be used.

The fluorination reaction is preferably carried out to attain a conversion of about 50% or higher, preferably, about 90% or higher. Conversion is calculated by the number of moles of reactant (HCO-1230xa) consumed divided by number of moles of reactant (HCO-1230xa) fed to the reactor multiplied by 100. The selectivity for HCFO-1233xf attained is preferably about 60% or higher and more preferably about 80% or higher. Selectivity is calculated by number of moles of product (HCFO-1233xf) formed divided by number of moles of reactant consumed.

This first step of the reaction may be conducted in any reactor suitable for a vapor phase fluorination reaction. In certain embodiments, the reactor is constructed from materials which are resistant to the corrosive effects of hydrogen fluoride and catalyst such as Hastalloy, Nickel, Incoloy, Inconel, Monel and fluoropolymer linings. The vessel is a fixed catalyst bed or fluidized bed. If desired, inert gases such as nitrogen or argon may be employed in the reactor during operation.

In general, the effluent from the fluorination reaction step, including any intermediate effluents that may be present in multi-stage reactor arrangements, may be processed to achieve desired degrees of separation and/or other processing. For example, in embodiments in which the reactor effluent comprises HCFO-1233xf, the effluent will generally also include HCl and one or more of HF, 2,3-dichloro-3,3-difluoropropene (HCFO-1232xf), 1,2-dichloro-3,3,3-trifluoropropene (HCFO-1223xd), trichlorofluoropropene (HCFO-1231) isomers, 2-chloro-1,1,1,2-tetrachloropropane (HCFC-244bb), and unreacted HCO-1230xa. Some portion or substantially all of these components of the reaction product may be recovered from the reaction mixture via any separation or purification method known in the art such as neutralization and distillation. It is expected that unreacted HCO-1230xa and HF could be recycled, completely or partially, to improve the overall yield of the desired HCFO-1233xf. HCFO-1232xf and any HCFO-1231 formed may also be recycled.

Optionally, hydrogen chloride is then recovered from the result of the fluorination reaction. Recovering of hydrogen chloride is conducted by conventional distillation where it is removed from the distillate. Alternatively, HCl can be recovered or removed by using water or caustic scrubbers. When a water extractor is used, HCl is removed as an aqueous solution. When caustic scrubbers are used, HCl is just removed from system as a chloride salt in aqueous solution.

In the second step of the process for forming 2,3,3,3-tetrafluoroprop-1-ene, HCFO-1233xf is converted to 2-chloro-1,1,1,2-tetrafluoropropane (HCFC-244bb). In one embodiment, this step may be performed in the liquid phase in a liquid phase reactor, which may be TFE or PFA-lined. Such a process may be performed in a temperature range of about 70-120° C. and about 50-120 psig.

Any liquid phase fluorination catalyst may be used in the invention. A non-exhaustive list includes Lewis acids, transition metal halides, transition metal oxides, Group IVb metal halides, a Group Vb metal halides, or combinations thereof. Non-exclusive examples of liquid phase fluorination catalysts are an antimony halide, a tin halide, a tantalum halide, a titanium halide, a niobium halide, and molybdenum halide, an iron halide, a fluorinated chrome halide, a fluorinated chrome oxide or combinations thereof. Specific non-exclusive examples of liquid phase fluorination catalysts are SbCl₅, SbCl₃, SbF₅, SnCl₄, TaCl₅, TiCl₄, NbCl₅, MoCl₆, FeCl₃, a fluorinated species of SbCl₅, a fluorinated species of SbCl₃, a fluorinated species of SnCl₄, a fluorinated species of TaCl₅, a fluorinated species of TiCl₄, a fluorinated species of NbCl₅, a fluorinated species of MoCl₆, a fluorinated species of FeCl₃, or combinations thereof. Antimony pentachloride is most preferred.

These catalysts can be readily regenerated by any means known in the art if they become deactivated. One suitable method of regenerating the catalyst involves flowing a stream of chlorine through the catalyst. For example, from about 0.002 to about 0.2 lb per hour of chlorine can be added to the liquid phase reaction for every pound of liquid phase fluorination catalyst. This may be done, for example, for from about 1 to about 2 hours or continuously at a temperature of from about 65° C. to about 100° C.

This second step of the reaction is not necessarily limited to a liquid phase reaction and may also be performed using a vapor phase reaction or a combination of liquid and vapor phases, such as that disclosed in U.S. Published Patent Application No. 20070197842, the contents of which are incorporated herein by reference. To this end, the HCFO-1233xf containing feed stream is preheated to a temperature of from about 50° C. to about 400° C., and is contacted with a catalyst and fluorinating agent. Catalysts may include standard vapor phase agents used for such a reaction and fluorinating agents may include those generally known in the art, such as, but not limited to, hydrogen fluoride.

In the third step of HFO-1234yf production, the HCFC-244bb is fed to a second vapor phase reactor (dehydrochlorination reactor) to be dehydrochlorinated to make the desired product 2,3,3,3-tetrafluoroprop-1-ene (HFO-1234yf). This reactor contains a catalyst that can catalytically dehydrochlorinate HCFC-244bb to make HFO-1234yf. The catalysts may be metal halides, halogenated metal oxides, neutral (or zero oxidation state) metal or metal alloy, or activated carbon in bulk or supported form. Metal halide or metal oxide catalysts may include, but are not limited to, mono-, bi-, and tri-valent metal halides, oxides and their mixtures/combinations, and more preferably mono-, and bi-valent metal halides and their mixtures/combinations. Component metals include, but are not limited to Cr³⁺, Fe³⁺, Mg2+, Ca²⁺, Ni²⁺, Zn²⁺, Pd²⁺, Li⁺, Na⁺, K⁺, and Cs⁺. Component halogens include, but are not limited to, F⁻, Cl⁻, Br⁻, and I⁻. Examples of useful mono- or bi-valent metal halide include, but are not limited to, LiF, NaF, KF, CsF, MgF₂, CaF₂, LiCl, NaCl, KCl, and CsCl. Halogenation treatments can include any of those known in the prior art, particularly those that employ HF, F₂, HCl, Cl₂, HBr, Br₂, HI, and I₂ as the halogenation source.

When neutral, i.e., zero valent, metals, metal alloys and their mixtures are used. Useful metals include, but are not limited to, Pd, Pt, Rh, Fe, Co, Ni, Cu, Mo, Cr, Mn, and combinations of the foregoing as alloys or mixtures. The catalyst may be supported or unsupported. Useful examples of metal alloys include, but are not limited to, SS 316, Monel 400, Inconel 825, Inconel 600, and Inconel 625.

Preferred, but non-limiting, catalysts include activated carbon, stainless steel (e.g. SS 316), austenitic nickel-based alloys (e.g. Inconel 625), nickel, fluorinated 10% CsCl/MgO, and 10% CsCl/MgF₂. The reaction temperature is preferably about 300-550° C. and the reaction pressure may be between about 0-150 psig. The reactor effluent may be fed to a caustic scrubber or to a distillation column to remove the by-product of HCl to produce an acid-free organic product which, optionally, may undergo further purification using one or any combination of purification techniques that are known in the art.

The present inventors have found that the presence of moisture in the compound of Formula I, for example, HCO-1230xa, or composition containing same causes problems. As provided herein, such moisture promotes the formation of oxidized oligomers of HCO-1230xa, which results in catalyst deactivation by blocking catalyst active sites. In addition, because HF is a raw material in the reaction, the moisture accelerates the corrosion of process lines and ultimately the formation of solid inorganic salts, which may land on the catalyst surface and also cause catalyst deactivation. For example, without wishing to be bound, when the compound of Formula I is HCO-1230xa, the organic byproduct in the first fluorination step is a pentanone and/or methylhexahydropentalene-1,6-dione. In addition, the presence of moisture can cause corrosion of the equipment used in the fluorination step and or/plugging of various equipment used in the fluorination, such as a vaporizer. Higher moisture content in the compound of Formula I, such as HCO-1230xa, or composition containing same, exacerbates these adverse effects. As the moisture content of the compound of Formula I, such as HCO-1230xa, or composition containing same is decreased, the efficiency of the vapor phase fluorination reaction (the first fluorination reaction) described herein is enhanced, and the catalyst life is lengthened, while decreasing the formation of side products that interferes with the efficiency of the fluorination reaction and decreases the catalyst life. By providing a feed stream that is substantially free from moisture or water, the catalyst life is extended and adverse affects of its presence are minimized, if not prevented. For example, even at concentrations of 100 ppm or less of water, as described in the examples hereinbelow, the catalyst life of the catalyst used in the vapor phase fluorination process described herein is enhanced relative to the catalyst life in the process when the compound of Formula I, such as HCO-1230xa, or composition containing same is conducted wherein the moisture content is not decreased. Moreover, if the moisture content is 100 ppm or less, it takes longer to plug up the vaporizer, if at all or corrode the equipment, such as the pipes, when the compound of formula I, e.g., HCO-1230xa or composition containing same were used without reducing the moisture content.

Unless indicated to the contrary, the terms “moisture” and “water” are treated as synonymous and are used interchangeably.

The following are examples of the invention and are not to be construed as limiting.

### EXAMPLES

### Example 1

The HCO-1230xa feed used in Example 1 had a purity of 99.2 GC (gas chromatogram) area % and contained 100 ppm of moisture.

A continuous vapor phase fluorination reaction system consisting of N₂, HF, and organic feed systems, feed vaporizer, superheater, 2 inch ID Monel reactor, acid scrubber, drier, and product collection system was used to study the reaction. The reactor was loaded with 1.8 liters of fluorinated Cr₂O₃ catalyst. The reactor was then heated to a temperature of about 180° C. with a N₂ purge going over the catalyst after the reactor had been installed in a constant temperature sand bath. HF feed was introduced to the reactor (via the vaporizer and superheater) as a co-feed with the N₂ for 15 minutes when the N₂ flow was stopped. The HF flow rate was adjusted to 1.9 lb/hr and then 1,1,2,3-tetrachloropropene (HCO-1230xa) feed was started to the reactor (via the vaporizer and superheater). The HCO-1230xa feed contained 5 ppm of di-isopropyl amine. The feed rate of HCO-1230xa was kept steady at 1.7 lb/hr and HF feed was kept steady at 3.2 lb/hr for about a 17 to 1 mole ratio of HF to HCO-1230xa. Once the reaction started the catalyst bed temperature rose to about 200° C. The reaction temperature was gradually increased as catalyst deactivation occurred to maintain desired product collection rate. The reaction pressure was kept constant at 100 psig. The reaction was able to run for about 180 hours. After about 180 hours on stream, then some problems arose; the vaporizer was severely plugged and the reaction was forced to be stopped. Solid material was recovered and analyzed by ICP and IC after being digested in a mixture of phosphoric acid and sulfuric acid and then diluted with DI water. As shown in Table 1, the majority of solid material (>70 wt %) is composed of inorganic salts. Most of the metals in the salts are originated from Monel tube/pipes, and the amount of metal fluorides is a lot more than that of metal chloride. These results indicate corrosion of Monel tubes/pipes had occurred, which is promoted by the presence of moisture. However, the reaction is able to run longer than if the HCO-1230xa feed contained more than 400 ppm of moisture.

TABLE 1
Compositions of solid material recovered from vaporizer

| Component | Wt % |
|---|---|
| Cr | 0.6 |
| Cu | 12.9 |
| Fe | 1.6 |
| Mn | 0.4 |
| Ni | 27.4 |
| K | 2.8 |
| Si | 0.3 |
| F⁻ | 22.0 |
| Cl⁻ | 4.5 |
| Total | 72.5* |

*The balance is mainly composed of polymer.

### Example 2

HCO-1230xa feed used in Example 2 had a purity of 99.2 GC (gas chromatogram) area % and contained 100 ppm of moisture.

A system consisting of N₂, HF, and organic feed systems, steam vaporizer, ¾″ OD U-shaped super-heater (immersed in sandbath), and acid scrubber was used for study. The U-shaped super-heater was heated to a temperature of about 180° C. in N₂ flow. HF and HCO-1230xa were introduced to the steam vaporizer and then the U-shaped super-heater at feed rates of 2.0 lb/h and 1.0 lb/h, respectively. The HCO-1230xa feed contained 5 ppm of di-isopropyl amine. The pressure in U-shaped super-heater was then built to 70 psig. After eight hours, the entire process stream from the U-shaped super-heater was directed to a DIT (Dry Ice Trap) and was collected for 15 minutes. 50 ml of CH₂Cl₂ and 530 ml DI H₂O were then sucked into DIT. The content of DIT was transferred into a Sep funnel for phase separation after being defrosted. A fraction of the separated organic phase was subject to non-volatile residual (NVR) determination. 347 ppm NVR was obtained. The NVR sample was then subject to ¹H-NMR and GC-MS analyses after being dissolved in methylene chloride. The ¹H-NMR analysis suggests the presence of long chain aliphatic hydrocarbon, which is possibly terminated as an organic acid (C═O, 1709-1730 cm⁻¹), and the GC-MS analysis indicates the presence of pentanone and methylhexahydropentalene-1,6-Dione, both of which contain oxygen atom. However, the amount of pentanone and methylhexahydripentalene-1,6-dione is less than that amount that would have been formed if the HCO-1230xa feed had a moisture content of 600 ppm.

### Example 3

This example illustrates the effectiveness of 3 A molecular sieves for removing moisture from HCO-1230xa feed. HCO-1230xa feed used in Example 1 had a purity of 99.2 GC (gas chromatogram) area % and contained 100 ppm of moisture. The HCO-1230xa feed contained 5 ppm of di-isopropyl amine. The HCO-1230xa feed was passed through a 2″ ID column loaded with 2 liters of 3 A molecular sieves at rate of 1.0 lb/h and sample was taken from a sampling port after drying column. Moisture level was determined to be 12 ppm using Mitsubishi Moisture Meter (Model CA-100), indicating 3 A molecular sieve is an effective drying agent for HCO-1230xa.

### Example 4

This example illustrates the performance of fluorinated Cr₂O₃ catalyst during the continuous vapor phase fluorination reaction of 1,1,2,3-tetrachloropropene (HCO-1230xa) to 2-chloro-3,3,3-trifluoropropene (HCFO-1233xf) with an HCO-1230xa feed containing 50 ppm of moisture.

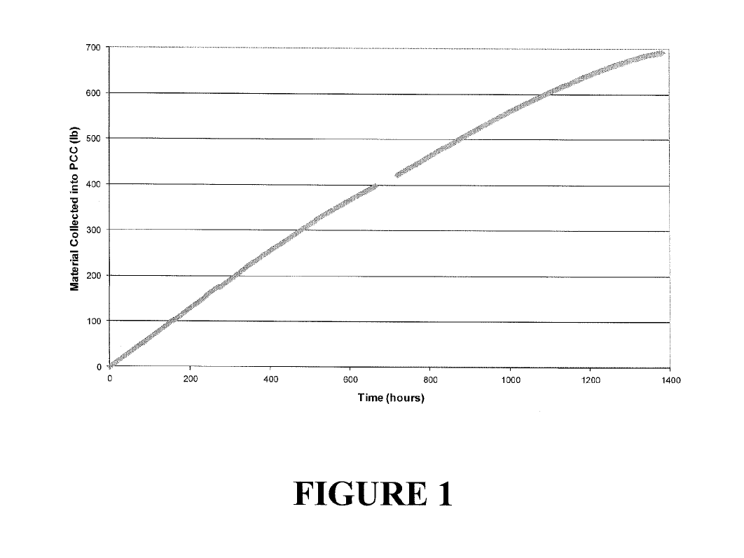

A continuous vapor phase fluorination reaction system consisting of N₂, HF, and organic feed systems, feed vaporizer, superheater, 2 inch ID Monel reactor, acid scrubber, drier, and product collection system was used to study the reaction. The reactor was loaded with 1.8 liters of fluorinated Cr₂O₃ catalyst. The reactor was then heated to a temperature of about 180° C. with a N₂ purge going over the catalyst after the reactor had been installed in a constant temperature sand bath. HF feed was introduced to the reactor (via the vaporizer and superheater) as a co-feed with the N₂ for 15 minutes when the N₂ flow was stopped. The HF flow rate was adjusted to 1.9 lb/hr and then 1,1,2,3-tetrachloropropene (HCO-1230xa) feed was started to the reactor (via the vaporizer and superheater) at a feed rate of 1.0 lb/hr for about a 17 to 1 mole ratio of HF to HCO-1230xa. The HCO-1230xa feed contained 5 ppm of di-isopropyl amine. Once the reaction started the catalyst bed temperature rose to about 200° C. due to the exothermic nature of the reaction. The reaction temperature (hot spot temperature) was gradually increased as catalyst deactivation occurred to maintain desired product collection rate. The reaction was stopped when reaction temperature reached about 300° C. In total, the reaction was run for about 1380 hours without any plug issue and about 690 lb of 99+% HCFO-1233xf was collected. The amount of product collected in the Product Collection Cylinder (PCC weight gain) as a function of time on stream is depicted in FIG. 1.

### Comparative Example

This example is prophetic. In this example, the feed system contains HCO-1230xa feed contains greater than 400 ppm of moisture. A continuous vapor phase fluorination reaction system consisting of N₂, HF, and organic feed systems, feed vaporizer, superheater, 2 inch ID Monel reactor, acid scrubber, drier, and product collection system is used to study the reaction. The reactor is loaded with 1.8 liters of fluorinated Cr₂O₃ catalyst. The reactor is then heated to a temperature of about 180° C. with a N₂ purge going over the catalyst after the reactor had been installed in a constant temperature sand bath. HF feed is introduced to the reactor (via the vaporizer and superheater) as a co-feed with the N₂ for 15 minutes and then the N₂ flow is stopped. The HF flow rate is adjusted to 1.9 lb/hr and then 1,1,2,3-tetrachloropropene (HCO-1230xa) feed is started to the reactor (via the vaporizer and superheater). The feed rate of HCO-1230xa is kept steady at 1.7 lb/hr and HF feed is kept steady at 3.2 lb/hr for about a 17 to 1 mole ratio of HF to HCO-1230xa. Once the reaction starts, the catalyst bed temperature will rise to about 200° C. The reaction temperature is gradually increased as catalyst deactivation occurs to maintain desired product collection rate. The vaporizer becomes severely plugged and the reaction is forced to be stopped in significantly less time than 180 hours. Further, the higher moisture content of HCO-1230xa causes the corrosion of Monel tubes/pipes to occur significantly earlier than in Example 1. Moreover, significantly more of the long chain aliphatic hydrocarbon than that produced in Example 2 is collected.

The above preferred embodiments and examples were given to illustrate the scope and spirit of the present invention. These embodiments and examples will make apparent to those skilled in the art other embodiments and examples. The other embodiments and examples are within the contemplation of the present invention. Therefore, the present invention should be limited only by the amended claims.

1. A feed stock for use in preparing a fluororolefin comprising;
a composition comprising 1,1,2,3-tetrachloropropene that is substantially free of water.

2. The feed stock of claim 1, wherein the composition comprises less than about 200 ppm of water.

3-4. (canceled)

5. A process for preparing 2-chloro-3,3,3-trifluoropropene comprising:
providing a starting composition comprising at least one compound of formula I
CX₂═CCl—CH₂X (I)
wherein X is independently selected from F, Cl, Br, and I, provided that at least one X is not fluorine and wherein the starting composition is substantially free of water; and
contacting said starting composition with a fluorinating agent to produce a final composition comprising 2-chloro-3,3,3-trifluoropropene.

6. The process of claim 5, wherein at least one compound of formula I is a compound comprising at least one X is a chlorine.

7. The process of claim 5, wherein at least one compound of formula I is a compound where all Xs are chlorine.

8. The process of claim 5, wherein the at least one compound of formula I comprises 1,1,2,3-tetrachloropropene.

9. The process of claim 5, wherein the contacting of said starting composition with a fluorinating agent occurs in a vapor phase.

10. The process of claim 5, wherein the contacting occurs in the presence of a catalyst.

11. The process of claim 10, wherein the catalyst is a vapor phase catalyst.

12. The process of claim 11, wherein the vapor phase catalyst is selected from the group consisting of a chromium oxide, a chromium hydroxide, a chromium halide, a chromium oxyhalide, an aluminum oxide, an aluminum hydroxide, an aluminum halide, an aluminum oxyhalide, a cobalt oxide, a cobalt hydroxide, a cobalt halide, a cobalt oxyhalide, a manganese oxide, a manganese hydroxide, a manganese halide, a manganese oxyhalide, a nickel oxide, a nickel hydroxide, a nickel halide, a nickel oxyhalide, an iron oxide, an iron hydroxide, an iron halide, an iron oxyhalide, inorganic salts thereof, fluorinated derivatives thereof and combinations thereof.

13. The process of claim 11 wherein the catalyst is a chromium oxide.

14. (canceled)

15. The process of claim 5, wherein the starting composition comprised of CX₂═CCl—CH₂X is made substantially free of water by distilling out water from said composition.

16. The process of claim 5, wherein the starting composition comprised of CX₂═CCl—CH₂X is made substantially free of water by contacting the starting composition comprised of CX₂═CCl—CH₂X with one or more desiccants for a time sufficient to reduce the concentration of water associated with the starting composition to a concentration that is substantially free of water.

17. The process of claim 16, wherein the dessicant(s) is selected from the group consisting of silica gel, activated charcoal, calcium sulfate, calcium chloride, montmorillonite clay, a molecular sieve, and combinations thereof.

18. The process of claim 5, wherein the starting composition comprises less than about 200 ppm of water.

19-20. (canceled)

21. A process for preparing 2,3,3,3-tetrafluoroprop-1-ene comprising:
providing a starting composition comprising a compound of formula I
CX₂═CCl—CH₂X (I)
wherein X is independently selected from F, Cl, Br, and I, provided that at least one X is not fluorine and the starting composition is substantially free of water;
contacting said starting composition with a first fluorinating agent to produce a first intermediate composition comprising 2-chloro-3,3,3-trifluoropropene;
contacting said first intermediate composition with a second fluorinating agent to produce a second intermediate composition comprising 2-chloro-1,1,1,2-tetrafluoropropane; and
dehydrochlorinating at least a portion of said 2-chloro-1,1,1,2-tetrafluoropropane to produce a reaction product comprising 2,3,3,3-tetrafluoroprop-1-ene.

22. The process The process of claim 21, wherein the starting composition comprises less than about 200 ppm of water.

23-24. (canceled)

25. The process of claim 21, wherein at least one compound of formula I is a compound comprising at least one X is a chlorine.

26. The process of claim 21, wherein at least one compound of formula I is a compound where all Xs are chlorine.

27. The process of claim 21, wherein the at least one compound of formula I is 1,1,2,3-tetrachloropropene.
